(19) United States
(12) Patent Application Publication
(10) Pub. No.: US 2015/0231245 A1
(43) Pub. Date: Aug. 20, 2015

(54) METHOD FOR ELIMINATING BIOFILM AND OTHER MEDICAL BENEFITS

(71) Applicant: Ben Oratz, Southbury, CT (US)

(72) Inventor: Ben Oratz, Southbury, CT (US)

(21) Appl. No.: 14/621,607

(22) Filed: Feb. 13, 2015

Related U.S. Application Data
(60) Provisional application No. 61/941,284, filed on Feb. 18, 2014.

(51) Int. Cl. A61K 41/00; A01N 59/00; A61K 33/00

(52) U.S. Cl. CPC A61K 41/0052; A61K 33/00; A01N 59/00

(57) ABSTRACT

A method of retarding the formation of biofilm, such as dental plaque biofilm on surfaces in the mouth, is provided. The method includes passing a liquid through a vessel having a magnetic field, ingesting the liquid, and then allowing the liquid to contact surfaces within the mouth for a predetermined amount of time to prevent or reduce the formation of dental plaque biofilm on the surfaces.

### CROSS-REFERENCE TO RELATED APPLICATION

This application claims the benefit of provisional patent application Ser. No. 61/941,284 filed in the United States Patent and Trademark Office on Feb. 18, 2014, the entire disclosure of which is incorporated by reference herein.

### STATEMENT REGARDING FEDERALLY SPONSORED RESEARCH DEVELOPMENT

Not applicable.

### BACKGROUND OF THE INVENTION

1. Field of the Invention

This invention relates to biofilm, and more particularly, to a method for retarding the formation of biofilm on a surface in the human body, such as dental plaque on teeth in the mouth. The method includes passing a liquid solution through a vessel having a magnetized field, ingesting the liquid solution, and then allowing the liquid solution to contact surfaces within the mouth for sufficient time to enable retardation of dental plaque biofilm on the surfaces.

2. Description of the Related Art

Up to 60% of the adult human body is made up of water. Each day humans must consume a certain amount of water to survive. Water is vital to all of our biological functions including circulation, digestion, absorption, and cleansing. On average, health experts recommend that humans drink between one and seven liters of water each day. Since drinking water is so vital to our health, the type and quality of the water we consume is important.

The present invention is focused on the advantageous biological effects that occur in people who consume fluids, namely water, which has been treated by exposure to magnetic fields for short time durations. For example, William H. Philpott, MD. and Sharon Toplin in their book Biomagnetic Handbook—A Guide to Medical Magnetics The Energy Medicine of Tomorrow discuss the therapeutic effects of magnetic fields on the body and specifically refer to the beneficial effects of drinking water which has been magnetically treated by placing a glass container of water on or near a magnet for a minimum of five minutes. In other published articles, Dr. Philpott discusses the role of magnetically treated oxygen in health maintenance.

The present inventor has realized a highly effective means of eliminating or reducing the formation of biofilm in the body, most particularly in the mouth, through the ingestion of water passed through a magnetic field using vessels such as the ones disclosed in U.S. Pat. Nos. 5,542,562 and 5,813,557, which are herein incorporated by reference in their entirety. Various other means of magnetically treating water are disclosed in U.S. Pat. No. 5,113,751 to Holcomb et al. and in other magnetic water treatment device patents discussed therein. The water treated by exposure to a magnetic field is fresher, more natural, bettering tasting and have more energy.

A biofilm is any group of microorganisms in which cells stick to each other on a surface. One such biofilm is dental plaque. Dental plaque is an oral biofilm that adheres to the teeth and consists of many species of both fungal and bacterial cells, salivary polymers and microbial extracellular products. These microorganisms first bind tightly together through London dispersion forces or van der Waals forces and ultimately create dental disease, such as tooth decay. Dental disease and dental biofilm are commonly created in the mouth, when the mouth is acidic in nature, hence having a low pH. The present inventor teaches that by drinking water that has been treated by being passed through a magnetic field, the van der Waals forces which bind the biofilm together are mitigated, and the formation of biofilm in the mouth can be prevented or eliminated. By drinking treated water, the elevated pH of the water inherently contains a strong oxidizing agent, which stabilizes the pH of the mouth and eliminates bacteria in the patient's oral biofilm. Further, drinking water with an elevated pH is beneficial for reducing water's surface tension by declustering H₂O molecules, which increases the bioavailability of water.

The present method further teaches that consuming water exposed to magnetic fields infuses energy into the body. Cells of the human body have been shown to positively respond to this infusion of energy. As the cells gain more electrical conductivity and kinetic energy they are able to absorb more nutrients and eliminate more waste. As the cells become more balanced, cell, tissue and organ functions are restored with healthier results. The health benefits realized include slowing down the damaging and aging of cells, reducing calcification in the body, which is beneficial for arteries, veins, organs and tissues, and increasing electrical conductivity, which in turn creates more energy, less fatigue and increases blood flow.

The water of the present invention has a beneficial effect on the blood brain barrier. Water is a natural best solvent that is not harmful to the brain and provides a method for administering methods for neurological diseases.

Plaque in the mouth may lead to other negative health issues, including leading to heart disease and other negative effects on the body which may be improved using water of the present invention.

Plaque may also be found in the arteries, veins and ingesting the water of the present invention may help reduce plaque in these and other areas of the body.

The water of the present invention may be topically applied to bed sores and other skin issues on patients.

Drinking the water of the present invention may help reduce kidney stones, gallstones, and urethra due to calcium and acid build-up that creates stones in these areas of the body.

Drinking the water of the present invention may help fibrin, fibrosis, atheromas, thrombosis, renal acidosis, glycated hemoglobin and blood glucose.

Using water of the present invention may reduce the need for blood thinners.

It is, therefore, a primary object of the present invention to provide an improved method for retarding the growth of biofilm on a surface within the human body.

It is another object of the present invention to provide an improved method that includes exposing liquid to a magnetic field, ingesting the liquid into the body, and allowing the liquid to contact surfaces within the human body.

It is another object of the present invention to provide a method of reducing or eliminating the growth of dental plaque biofilm in the mouth, specifically on the teeth and gums.

It is another object of the present invention to provide a method which utilizes a vessel, such as a container or conduit in which a liquid is exposed to a magnetic field.

It is another object of the present invention to provide a method which utilizes a vessel for a fluid such as water, oxygen or a liquid to be intravenously injected into the body.

It is another object of the present invention to provide a method which includes applying liquid to a surface within the body by brushing, flossing, drinking, painting, sponging, rinsing, soaking, washing, and contacting the surface for retarding the growth of biofilm on the surface.

It is another object of the present invention to provide a method which utilizes a vessel where magnets are surrounded by a metallic band or collar to enhance the magnetic effect.

It is another object of the present invention to provide a method, which retards Xerostomia, commonly known as acidic mouth, by ingesting liquid that has been exposed to a magnetic field.

It is another object of the present invention to provide a method, which retards the growth of biofilm in the body, such as the wall of the intestine, through the ingestion of liquid that has been exposed to a magnetic field.

It is another object of the present invention to provide a method, which retards metabolic acidosis by increasing the pH in the body's blood, through the ingestion of liquid that has been exposed to a magnetic field.

It is another object of the present invention to provide a method, which increases the alkalinity in the body to reduce, uric acid, galls stones, kidney stones, gum disease, and calcium deposits through the ingestion of liquid that has been exposed to a magnetic field.

It is another object of the present invention to provide a method, which retards the build-up of plaque in arteries and thereby reduces atheroma, thrombosis, deep vein thrombosis, and lung thrombosis, through the ingestion of liquid that has been exposed to a magnetic field.

It is another object of the present invention to provide a method, which increases electrical conductivity in the body, and induces more energy, less fatigue and increase blood flow through the ingestion of liquid that has been exposed to a magnetic field.

It is another object of the present invention to provide a method, which creates additional kinetic energy in the body thereby retarding DNA damage in the kidneys, liver and blood through the ingestion of liquid that has been exposed to a magnetic field.

It is another object of the present invention to provide a method, which treats disease, such as diabetes and cancer, by increasing metabolism and improving bioactivation through the ingestion of liquid that has been exposed to a magnetic field.

It is another object of the present invention to provide a method, which reduces obesity through the ingestion of liquid that has been exposed to a magnetic field.

It is another object of the present invention to utilize a more convenient and easier means for applying liquid to the body capable of providing direct prolonged treatment.

### BRIEF SUMMARY OF THE INVENTION

In accordance with one aspect of the present invention, a method of retarding the formation of biofilm on a surface within the human body is provided. The method first includes exposing a liquid to a magnetic field. Then the method includes applying the liquid to at least one surface with the body for a predetermined amount of time to reduce the formation of biofilm on the surface.

The method may further include passing the liquid through a vessel having a magnetic field. The method may further comprise ingesting the liquid after the liquid is passed through a vessel having a magnetic field. The method may further comprise intravenously injecting liquid into the body after it has passed through a vessel having a magnetic field.

The liquid of the method may comprise an agent that inhibits the growth of biofilm.

The method may further comprise re-applying the liquid to at least one surface within the body for a predetermined amount of time to reduce the formation of biofilm on the surface.

The biofilm may comprise bacteria associated with plaque formed on surfaces of teeth in the mouth.

The human body includes blood with a pH, such that after the liquid is ingested, the pH of the blood increases and effectively diminishes acidosis within the human body.

The method may further comprise exposing cells, tissues and organs of the human body to the liquid after the liquid passes through the magnetic field for decreasing aging effects of cells, tissues and organs.

The method may further comprise exposing tissues and organs of the human body to the liquid for reducing calcification in tissues and organs.

In accordance with an additional embodiment a method of retarding the formation of biofilm, such as dental plaque biofilm on surfaces in the mouth is provided. The method includes passing a liquid through a vessel having a magnetic field, and then ingesting the liquid into the mouth. The method further includes allowing the liquid to contact the surfaces within the mouth for a predetermined time to reduce the formation of dental plaque biofilm on the surfaces.

The liquid may be water.

The liquid of the method may comprise an agent that inhibits the growth of biofilm.

The method may further comprise allowing the agent within the liquid to react with the biofilm on surfaces in the mouth for a predetermined amount of time, such that the biofilm is released from the surface.

In accordance with an additional embodiment a method of treating teeth and gums within the mouth for retarding the growth of dental plaque biofilm on the teeth and gums is provided. The method comprises applying to the teeth and gums for a predetermined amount of time liquid that was exposed to a magnetic field to reduce or prevent the formation of dental plaque biofilm on the teeth and gums. The liquid may be water. The method may further comprise reapplying to the teeth and gums for a predetermined amount of time liquid that was exposed to a magnetic field to reduce or prevent the formation of dental plaque biofilm on the teeth and gums. The method further comprising repeating the method on a daily basis for further preventing, reducing, or eliminating the formation of biofilm.

In accordance with an additional embodiment a method of treating a surface for retarding the growth of biofilm on the surface is provided. The method comprises applying to the surface for a predetermined amount of time liquid that was exposed to a magnetic field to reduce or prevent the formation of biofilm on the surface. The surface may be from the group consisting of medical instruments, medical equipment, and catheters.

### BRIEF DESCRIPTION OF THE SEVERAL VIEWS OF THE DRAWINGS

To these and to such other objects that may hereinafter appear, the present invention relates to an improved method of retarding the formation of biofilm as described in detail in the following specification and recited in the annexed claims, taken together with the accompanying drawings, in which like numerals refer to like parts in which:

FIG. 1 is a flow chart illustrating a method of retarding the formation of biofilm, such as dental plaque biofilm on surfaces in the mouth, in accordance with the preferred embodiment of the present invention; and

FIG. 2 is a flow chart illustrating a method of retarding the formation of biofilm from a surface within the human body in accordance with an additional embodiment of the present invention.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### DETAILED DESCRIPTION OF THE INVENTION

The present invention is an improved method for retarding the growth of biofilm on surfaces within the human body. In the preferred embodiment, the biofilm is dental plaque biofilm found on surfaces of the teeth and gums in the mouth. In alternate embodiments, the biofilm may be found on or within cells, tissues, or organs.

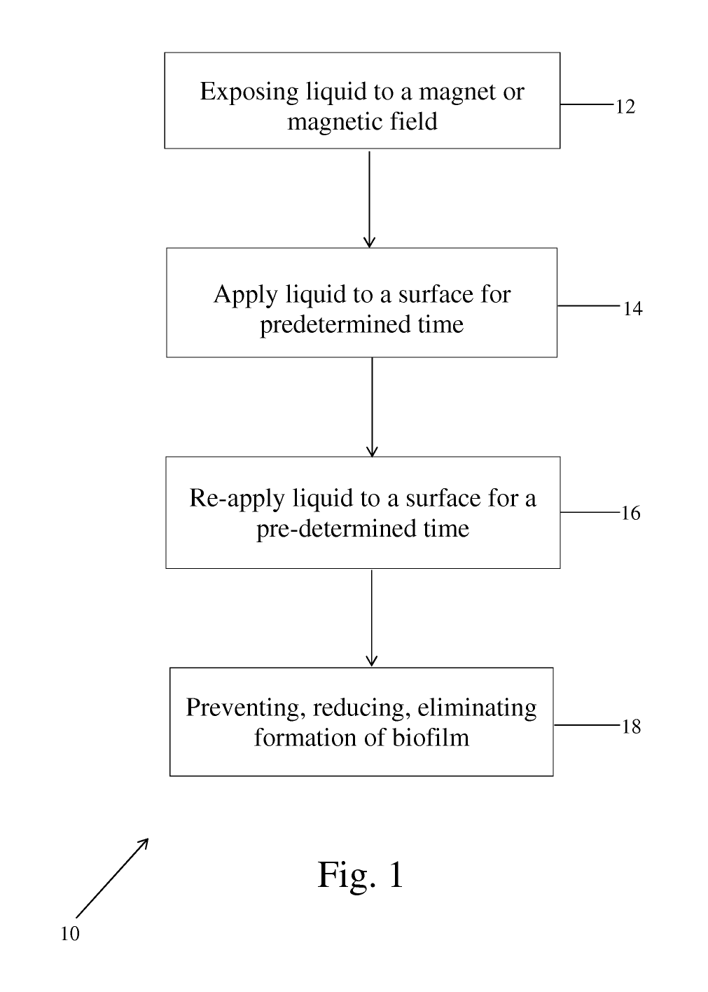

FIG. 1 illustrates the improved method 10 of the present invention. In the first step 12 of the preferred method, a liquid or fluid is exposed to a magnet or magnetic field. The liquid is preferably water, but other fluids or oxygen are contemplated. The water is preferably exposed to the magnetic field through the use of a unique vessel, such as the one disclosed in U.S. Pat. Nos. 5,542,562 and 5,813,557, which are herein incorporated by reference in their entirety. Preferably, the water is exposed to the magnetic field for a prolonged period of time. In additional embodiments, the liquid may include an agent that inhibits the growth of biofilm.

The second step 14 of the method involves applying the liquid directly to a surface within or on the body. The liquid is applied for a predetermined length of time. Preferably, the liquid is applied to the surface by ingesting through the mouth. In alternative embodiments, the liquid may be applied to the surface topically using one or more of the following a brush, sponge, floss, dropper or spoon. Alternatively, if practical, the surface may be submerged into the liquid or contacted directly with the liquid. In even further embodiments, the liquid may be injected within the body, blood or cells, using various known devices, including but not limited to needles or syringes.

The third step 16 of the method is optional and includes re-applying the liquid to the surface of the body, or injecting, submerging, contacting or exposing cells or other surfaces to the liquid for a predetermined length of time.

The fourth step 18 of the method involves allowing the liquid to react with the surface, in order to prevent, reduce, or eliminate the formation of biofilm on the surface. When biofilm is already present on the surface, the liquid will enable the release of the biofilm from the surface.

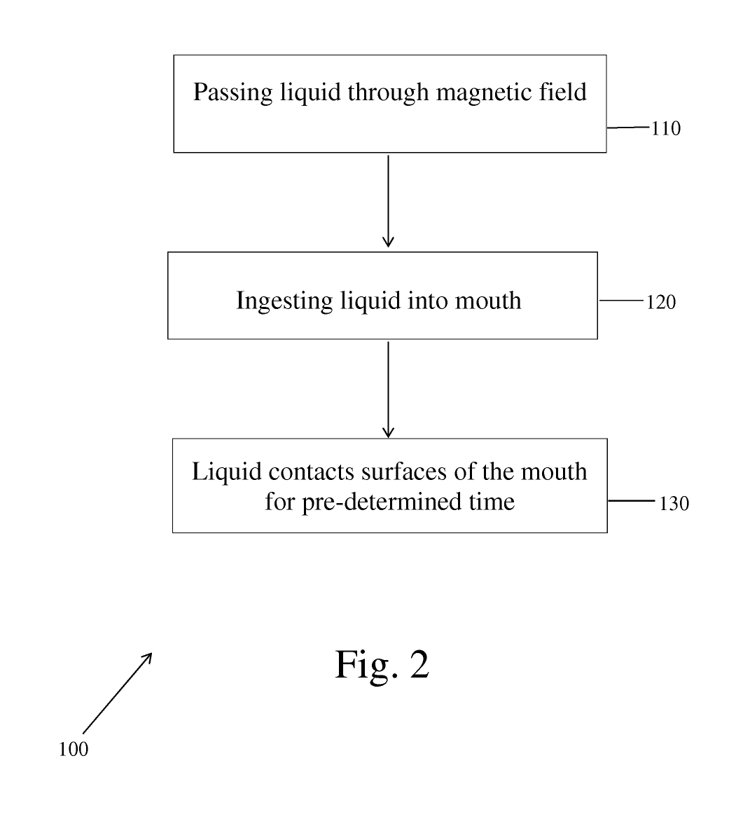

FIG. 2 illustrates a second method 100 of retarding the formation of biofilm from a surface within the human body in accordance with an additional embodiment of the present invention.

In the first step 110 of the second method, a liquid is exposed to a magnet or magnetic field. The liquid is preferably water. The water is preferably exposed to the magnetic field through the use of a unique vessel, such as the one discussed supra in U.S. Pat. Nos. 5,542,562 and 5,813,557. Preferably, the water is exposed to the magnetic field for a prolonged period of time.

In the second step 120, the liquid is ingested into the mouth. In the third step 130, the liquid is allowed to directly contact surfaces of the mouth, including teeth and gums, for a predetermined length of time. The liquid may be swooshed around in the mouth, used to brush or floss teeth, held in the mouth, gurgled or simply drank. When the liquid contacts the surfaces of the mouth, it reacts with the teeth and gums in order to prevent, reduce, or eliminate the formation of biofilm thereon. When biofilm is already present on the teeth and gums, the liquid will enable the release of the biofilm therefrom.

Repeating the method of the present invention on a daily basis further prevents, reduces, or eliminates the formation of biofilm.

It should be understood that numerous variations are possible, while adhering to the inventive concept. Such variations are contemplated as being a part of the present invention.

While only the preferred embodiment of the present invention has been disclosed for purposes of illustration, it is obvious that many modifications and variations could be made thereto. It is intended to cover all of those modifications and variations, which fall within the scope of the present invention as defined by the following claims.

I claim:

1. A method of retarding the formation of biofilm on a surface within the human body, the method comprising:
exposing a liquid to a magnetic field; and
applying the liquid to at least one surface within the body for a predetermined amount of time to reduce the formation of biofilm on said surface.

2. The method of claim 1, wherein the liquid passes through a vessel having a magnetic field.

3. The method of claim 2, further comprising ingesting the liquid after said liquid is passed through a vessel having a magnetic field.

4. The method of claim 2, further comprising intravenously injecting liquid into the body after it has passed through a vessel having a magnetic field.

5. The method of claim 1, wherein the liquid comprises an agent that inhibits the growth of biofilm.

6. The method of claim 1, further comprising re-applying the liquid to the at least one surface of the body for a predetermined amount of time to reduce the formation of biofilm on said surface.

7. The method of claim 1, wherein biofilm comprises bacteria associated with plaque formed on the surfaces of teeth in the mouth.

8. The method of claim 7, wherein the human body includes blood with a pH, such that after the liquid is ingested, the pH of the blood increases and effectively diminishes acidosis within the human body.

9. The method of claim 7, further comprising exposing cells, tissues and organs within the human body to the liquid after the liquid passes through the magnetic field for decreasing aging effects of cells, tissues and organs.

10. The method of claim 1, further comprising exposing cells within the human body to the liquid for reducing calcification in tissues and organs.

11. A method of retarding the formation of biofilm, such as dental plaque biofilm on surfaces in the mouth, the method comprising:
passing a liquid through a vessel having a magnetic field;
ingesting the liquid into the mouth; and
allowing the liquid to contact the surfaces within the mouth for a predetermined time to reduce the formation of dental plaque biofilm on said surfaces.

12. The method of claim 11, wherein the liquid is water.

13. The method of claim 11, wherein the liquid comprises an agent that inhibits the growth of biofilm.

14. The method of claim 13, further comprising allowing the agent within the liquid to react with the biofilm on surfaces in the mouth for a predetermined amount of time, such that the biofilm is released from the surface.

15. A method of treating teeth and gums within the mouth for retarding the growth of dental plaque biofilm on said teeth and gums, comprising applying to said teeth and gums for a predetermined amount of time liquid that was exposed to a magnetic field to reduce or prevent the formation of dental plaque biofilm on said teeth and gums.

16. The method of claim 15, wherein the liquid is water.

17. The method of claim 15, further comprising reapplying to said teeth and gums for a predetermined amount of time liquid that was exposed to a magnetic field to reduce or prevent the formation of dental plaque biofilm on said teeth and gums.

18. The method of claim 15, further comprising repeating the method on a daily basis for further preventing, reducing, or eliminating the formation of biofilm.

19. A method of treating a surface for retarding the growth of biofilm on said surface, comprising applying to said surface for a predetermined amount of time liquid that was exposed to a magnetic field to reduce or prevent the formation of biofilm on said surface.

20. The method of claim 19, wherein the surface is from the group consisting of medical instruments, medical equipment, and catheters.
